(19) United States
(12) Patent Application Publication
(10) Pub. No.: US 2026/0207442 A1
(43) Pub. Date: Jul. 23, 2026

(54) POWDER COSMETIC

(71) Applicant: SHISEIDO COMPANY, LTD., Tokyo (JP)

(72) Inventors: Shun ITO, Tokyo (JP); Megumi SAITO, Tokyo (JP); Mayu FUNAYAMA, Tokyo (JP); Toshihiko NAKANE, Tokyo (JP)

(73) Assignee: SHISEIDO COMPANY, LTD., Tokyo (JP)

(21) Appl. No.: 19/143,138

(22) PCT Filed: Jan. 12, 2024
(86) PCT No.: PCT/JP2024/000519

§ 371 Date: Jun. 25, 2025

(30) Foreign Application Priority Data
Jan. 25, 2023 (JP) 2023-009569

(51) Int. Cl. A61K 8/02; A61K 8/92; A61Q 1/12

(52) U.S. Cl. CPC A61K 8/022; A61K 8/92; A61Q 1/12

(57) ABSTRACT

A powder cosmetic including: (A) a powdered oil or fat containing from 80% to 99% by mass of one or more XXX-type triglycerides having a fatty acid residue X having x carbon atoms at positions 1 to 3 and from 20% to 1% by mass of one or more X2Y-type triglycerides in which one of the fatty acid residues X in the XXX-type triglycerides has been substituted with a fatty acid residue Y having y carbon atoms with respect to the total triglyceride content of 100% by mass, in which the number of carbon atoms x is an integer selected from 8 to 12 the number of carbon atoms y is each independently an integer selected from x+2 to x+8; a powder component (B) (excluding the powdered oil or fat (A)), and a liquid oil or fat (C) that is incompatible with the powdered oil or fat (A) is used.

### FIELD OF THE INVENTION

The present invention relates to a powder cosmetic, and in particular, to a loose powder.

### BACKGROUND ART

Conventionally, various cosmetics that can be applied to the skin to improve the skin texture have been investigated. Cosmetics of this kind are required to have various properties when used.

For example, Patent Literature 1 proposes a powdery cosmetic composition containing a pulverulent phase, a liquid fatty phase, and a film-forming polymer in order to improve the skin texture.

### CITATION LIST

### Patent Literature

Patent Literature 1: JP 2015-51957 A

### SUMMARY OF THE INVENTION

However, the powder cosmetic described in Patent Literature 1 has a strong powdery feel and does not spread well on the skin when applied. Therefore, there remains a demand for powder cosmetics that spread easily on the skin when applied and provide a smooth feel when used.

In general, when improving the desired properties of a cosmetic depending on its intended use, it becomes difficult to balance the other properties, making it difficult to simultaneously satisfy all the properties. Therefore, there remains a demand for cosmetics that achieve such a balance and simultaneously improve many of the properties.

As a result of intensive studies in order to solve the above-described problems, the present inventors have found that when a powdered oil or fat and a liquid oil or fat that is incompatible with the powdered oil or fat are blended with a powder cosmetic containing large amounts of powder components, a smooth feel on the skin when used upon application is realized, in addition to that, skin glow is improved as the powdered oil or fat dissolves on the skin, resulting in a very beautiful finish. This has led to the completion of the present invention.

According to the present invention, the following inventions are provided.
[1] A powder cosmetic comprising:
(A) a powdered oil or fat containing from 80% to 99% by mass of one or more XXX-type triglycerides having a fatty acid residue X having x carbon atoms at positions 1 to 3 and from 20% to 1% by mass of one or more X2Y-type triglycerides in which one of the fatty acid residues X in the XXX-type triglycerides is substituted with a fatty acid residue Y having y carbon atoms with respect to the total triglyceride content of 100% by mass, wherein the number of carbon atoms x is an integer selected from 8 to 12, and the number of carbon atoms y is each independently an integer selected from x+2 to x+8;
(B) a powder component (excluding the powdered oil or fat (A)); and
(C) a liquid oil or fat that is incompatible with the powdered oil or fat (A).
[2] The powder cosmetic according to [1], wherein the powdered oil or fat (A) contains from 90% to 99% by mass of an XXX-type triglyceride having a fatty acid residue X having x carbon atoms at positions 1 to 3 and from 10% to 1% by mass of one or more X2Y-type triglycerides in which one of the fatty acid residues X in the XXX-type triglyceride is substituted with a fatty acid residue Y having y carbon atoms with respect to the total triglyceride content of 100% by mass, the number of carbon atoms x is 10, and the number of carbon atoms y is each independently an integer selected from x+4 to x+8.
[3] The powder cosmetic according to [1] or [2], wherein the powdered oil or fat (A) has a melting point of from 25° C. to 50° C.
[4] The powder cosmetic according to any one of [1] to [3], wherein the content of the powdered oil or fat (A) is from 0.5% by mass to 20% by mass with respect to a total mass of the powder cosmetic.
[5] The powder cosmetic according to any one of [1] to [4], wherein the content of the liquid oil or fat (C) is from 0.1% by mass to 10% by mass with respect to a total mass of the powder cosmetic.
[6] The powder cosmetic according to any one of [1] to [5], wherein a blending ratio (A/C) of the powdered oil or fat (A) to the liquid oil or fat (C) is from 0.1 to 10 on a mass basis.
[7] The powder cosmetic according to any one of [1] to [6], wherein a total content of the powdered oil or fat (A) and the powder component (B) is from 90% by mass to 99.9% by mass with respect to a total mass of the powder cosmetic.
[8] The powder cosmetic according to any one of [1] to [7], wherein the liquid oil or fat (C) is a silicone oil.
[9] The powder cosmetic according to any one of [1] to [8], wherein the powder component (B) contains at least one selected from the group consisting of mica, synthetic phlogopite, silica, glass, magnesium myristate, lauroyl lysine, talc, synthetic phlogopite iron, a cellulose powder, a urethane powder, a silicone resin powder, a polymethacrylic acid powder, titanium oxide, iron oxide, and a tar dye.
[10] The powder cosmetic according to any one of [1] to [9], wherein the powder cosmetic is a loose powder.

According to the present invention, a powder cosmetic that is excellent in the smoothness upon application, the suppression of powdery feel upon application, and the skin glow after application is provided.

### DESCRIPTION OF EMBODIMENTS

### [Powder Cosmetic]

The powder cosmetic according to the present invention includes the powdered oil or fat (A), the powder component (B) (excluding the powdered oil or fat (A)), and the liquid oil or fat (C) that is incompatible with the powdered oil or fat (A) as essential components. The powder cosmetic according to the present invention may further contain other components depending on the purpose. The powder cosmetic according to the present invention can simultaneously improve the smoothness upon application, the suppression of powdery feel upon application, and the skin glow after application. Each component contained in the powder cosmetic according to the present invention will be described in detail below.

### (A) Powdered Oil or Fat

The powdered oil or fat according to the present invention is a solid fat that is in a powder form at room temperature (20° C.). The melting point of the powdered oil or fat is preferably from 25° C. to 50° C., more preferably from 25° C. to 45° C., still more preferably from 25° C. to 40° C., and even more preferably from 25° C. to 35° C. The melting point of the powdered oil or fat can be measured using a differential scanning calorimeter (DSC) by an ordinary method. The peak top value on the DSC chart is taken as the melting point. As long as the melting point of the powdered oil or fat falls within the above-described range, it will melt easily when applied to the skin, and will absorb heat from the skin, making it easier to feel a cool sensation.

The powdered oil or fat usually has a plate-like crystal or spherical crystal form, and preferably has a plate-like crystal form. The powdered oil or fat has an average particle size (effective diameter) of, for example, from 50 to 400 μm, preferably from 50 to 300 μm, more preferably from 50 to 250 μm, and still more preferably from 50 to 200 μm. Here, the average particle size (effective diameter) is a value (d50) measured by dry measurement based on the laser diffraction scattering method (ISO 133201, ISO 9276-1) using a particle size distribution measuring device (e.g., Microtrac MT3300ExII manufactured by NIKKISO CO., LTD.). The effective diameter means the particle diameter of a sphere when the actually measured diffraction pattern of a crystal to be measured fits a theoretical diffraction pattern obtained by assuming the crystal to be the sphere. In this way, in the case of the laser diffraction scattering method, the effective diameter is calculated by fitting the theoretical diffraction pattern obtained, assuming a spherical shape to the actually measured diffraction pattern. Thus, the same principle can be used for measurement whether the subject to be measured is a plate-like crystal or a spherical crystal.

The powdered oil or fat contains one or more XXX-type triglycerides having a fatty acid residue X having x carbon atoms at positions 1 to 3 and one or more X2Y-type triglycerides in which one of the fatty acid residues X in the XXX-type triglycerides is substituted with a fatty acid residue Y having y carbon atoms.

The fatty acid residue X may be a saturated or unsaturated fatty acid residue. The number of carbon atoms x of the fatty acid residue X in the XXX-type triglyceride is an integer selected from 8 to 12, and preferably 10. Specific examples of the fatty acid residue X include saturated fatty acid residues such as caprylic acid (C8:0), capric acid (C10:0), and lauric acid (C12:0), with capric acid being preferred. In addition, the number of carbon atoms y of the fatty acid residue Y in the X2Y-type triglyceride is each independently from x+2 to x+8, and preferably from x+4 to x+8. Specific examples of the fatty acid residue Y include saturated fatty acid residues such as myristic acid (C14:0), palmitic acid (C16:0), and stearic acid (C18:0), with myristic acid being preferred.

In a case in which the total triglyceride content is 100% by mass, the content of the XXX-type triglyceride is from 80% to 99% by mass and preferably from 90% to 99% by mass, and the content of the X2Y-type triglyceride is from 20% to 1% by mass and preferably from 10% to 1% by mass. It is considered that in a case in which the contents of the XXX-type triglyceride and the X2Y-type triglyceride are within the above-described ranges, as there is a small amount of an X2Y-type triglyceride which has one fatty acid with a long chain length, when the X2Y-type triglyceride is cooled from a molten state to crystallize, it is mixed into an oil or fat crystal consisting of an XXX-type triglyceride, and interrupts the continuous crystal growth of the XXX triglyceride, which result is a crystallized solid that is very loose (expanded in volume and filled with voids). The resulting solid has the form of an aggregate of powdered oil or fat crystallized in a very coarse state, is brittle and crumbles even upon slight impact, and easily assumes a powdery form.

A commercially available product can be used as the powdered oil or fat. It is preferable to use Enequick manufactured by The Nisshin OilliO Group, Ltd. as the powdered oil or fat.

For the method of producing a powdered oil or fat, Japanese Patent No. 5937771 can be referred to. Specifically, the powdered oil or fat can be produced by the following steps. The powdered oil or fat can be produced by a method comprising:
(a) a step of preparing an oil or fat composition containing from 80% to 99% by mass of an XXX-type triglyceride having a fatty acid residue X having x carbon atoms at positions 1 to 3 and from 20% to 1% by mass of an X2Y-type triglyceride in which one of the fatty acid residues X in the XXX-type triglyceride is substituted with a fatty acid residue Y having y carbon atoms with respect to the total triglyceride content of 100% by mass, wherein the number of carbon atoms x is an integer selected from 8 to 12, and the number of carbon atoms y is each independently an integer selected from x+2 to x+8;
(b) an optional step of heating the oil or fat composition to melt a triglyceride contained in the oil or fat composition, thereby obtaining the oil or fat composition in a molten state; and
(d) a step of cooling the oil or fat composition in a molten state, thereby obtaining a powdered oil or fat.

Between steps (b) and (d) above, an optional step (c) for promoting powder generation may be included, such as (c1) a seeding step, (c2) a tempering step, and/or (c3) a pre-cooling step. Furthermore, the powdered oil or fat obtained in step (d) above may be obtained by a step (e) of pulverizing a solid obtained after cooling in step (d), thereby obtaining a powdered oil or fat. The above-described steps (a) to (e) will be described below.

### (a) Step of Preparing Oil or Fat Composition I

The oil or fat composition prepared in step (a) contains the above-mentioned XXX-type triglyceride (one or more types) and X2Y-type triglyceride (one or more types) in the above-mentioned values in % by mass. Specifically, for example, the oil or fat composition is obtained via a step of obtaining an XXX-type triglyceride having a fatty acid residue X having x carbon atoms at positions 1 to 3 (one or more types) and a YYY-type triglyceride having a fatty acid residue Y having y carbon atoms at positions 1 to 3 (one or more types) separately, mixing the triglycerides in a mass ratio of XXX-type triglyceride/YYY-type triglyceride of from 90/10 to 99/1 to obtain a reactive substrate (the number of carbon atoms x is an integer selected from 8 to 12, and the number of carbon atoms y is an integer selected from x+2 to x+8), heating the reactive substrate, and carrying out a transesterification reaction in the presence of a catalyst.

### <Reactive Substrate>

First, the XXX-type triglyceride (one or more types) and the YYY-type triglyceride (one or more types) are mixed, thereby obtaining a reactive substrate. Here, details of the XXX-type triglyceride are as described above.

The YYY-type triglyceride is a triglyceride having a fatty acid residue Y having y carbon atoms at positions 1 to 3. Here, the number of carbon atoms y and the fatty acid residue Y are as described above.

The XXX-type triglyceride and the YYY-type triglyceride can also be obtained by direct synthesis using a fatty acid or a fatty acid derivative and glycerin. Taking the XXX-type triglyceride as an example, the method of directly synthesizing an XXX-type triglyceride can be: (i) a method of directly esterifying a fatty acid having X carbon atoms with glycerin (direct ester synthesis); (ii) a method of reacting a fatty acid alkyl (e.g., fatty acid methyl and fatty acid ethyl) in which a carboxyl group of a fatty acid X having x carbon atoms is bonded to an alkoxyl group with glycerin under basic or acidic catalytic conditions (ester exchange synthesis using a fatty acid alkyl); and (iii) a method of reacting a fatty acid halide (e.g., fatty acid chloride and fatty acid bromide) in which the hydroxyl group of a carboxyl group of a fatty acid X having x carbon atoms is substituted with a halogen with glycerin in the presence of a basic catalyst (acid halide synthesis).

The XXX-type triglyceride and the YYY-type triglyceride can be produced by any of the above-mentioned methods (i) to (iii). However, from the viewpoint of ease of production, (i) direct ester synthesis or (ii) transesterification synthesis using a fatty acid alkyl is preferred, and (i) direct ester synthesis is more preferred.

To produce an XXX-type triglyceride or a YYY-type triglyceride by the direct ester synthesis (i), it is preferable to use from 3 to 5 moles, and more preferably from 3 to 4 moles, of the fatty acid X or fatty acid Y per mole of glycerin from the viewpoint of production efficiency.

The reaction temperature in the direct ester synthesis (i) of the XXX-type triglyceride or YYY-type triglyceride may be any temperature at which the water produced by the esterification reaction can be removed from the system, and for example, it is preferably from 120° C. to 300° C., more preferably from 150° C. to 270° C., and still more preferably from 180° C. to 250° C. By carrying out the reaction at from 180° C. to 250° C., the XXX-type triglyceride or the YYY-type triglyceride can be produced particularly efficiently.

In the direct ester synthesis (i) of the XXX-type triglyceride or YYY-type triglyceride, a catalyst for promoting the esterification reaction may be used. Examples of the catalyst include an acid catalyst and an alkoxide of an alkaline earth metal. The amount of the catalyst used is preferably about from 0.001% to 1% by mass based on the total mass of the reaction raw materials.

In the direct ester synthesis (i) of the XXX-type triglyceride or YYY-type triglyceride, the catalyst and unreacted raw materials can be removed after the reaction by carrying out known purification treatments such as washing with water, alkali deoxidation and/or deoxidation under reduced pressure, and adsorption treatment. Furthermore, the reaction product obtained can be further purified via decolorization and deodorization treatment.

The XXX-type triglyceride and the YYY-type triglyceride are mixed based on the mass ratio of XXX-type triglyceride/YYY-type triglyceride, which is from 90/10 to 99/1, preferably from 93/7 to 99/1, and more preferably from 95/5 to 99/1. In particular, in a case in which the fatty acid residue X has 10 carbon atoms and the fatty acid residue Y has from 14 to 18 carbon atoms, the mass ratio of XXX-type triglyceride/YYY-type triglyceride is preferably from 95/5 to 99/1. In addition, in a case in which the fatty acid residue X has 12 carbon atoms and the fatty acid residue Y has from 16 to 18 carbon atoms, the mass ratio of XXX-type triglyceride/YYY-type triglyceride is preferably from 95/5 to 99/1.

### <Other Triglycerides>

Examples of triglycerides that can be raw materials for the reactive substrate may include the above-described XXX-type and YYY-type triglycerides as well as various triglycerides unless the effects of the present invention are impaired. Examples of other triglycerides can include X2Y-type triglycerides in which one fatty acid residue X in the above-described XXX-type triglyceride is substituted with a fatty acid residue Y and XY2-type triglycerides in which two fatty acid residues X in the above-described XXX-type triglyceride are substituted with a fatty acid residue Y.

The amount of the above-described other triglycerides is, for example, from 0% to 15% by mass, preferably from 0% to 7% by mass, and more preferably from 0% to 4% by mass when the total mass of the XXX-type triglyceride and the YYY-type triglyceride is 100% by mass.

In addition, a naturally derived triglyceride composition may be used in place of the above-described XXX-type triglyceride or YYY-type triglyceride. Examples of naturally derived triglyceride compositions include palm kernel oil, palm kernel olein, palm kernel stearin, rapeseed oil, coconut oil, soybean oil, sunflower oil, safflower oil, and palm stearin. These naturally derived triglyceride compositions may be hardened oils, partially hardened oils, or extremely hardened oils that have been further modified by hydrogenation or the like.

The amount of the above-described naturally derived triglyceride composition depends on the amount of the necessary XXX-type triglyceride or YYY-type triglyceride contained in these naturally derived triglyceride compositions. However, for example, in a case in which X in the XXX-type triglyceride is capric acid, and a palm kernel stearin hardened oil is used as the origin of the YYY-type triglyceride, it is appropriate that a triglyceride having Y residues at positions 1 to 3 contained in the palm kernel stearin hardened oil is contained as the above-described YYY-type triglyceride in a necessary amount, which means an amount that satisfies a mass ratio of XXX-type triglyceride/YYY-type triglyceride of from 90/10 to 99/1, preferably from 93/7 to 99/1, and more preferably from 95/5 to 98/2.

### <Other Components>

Examples of raw materials that constitute the reactive substrate may include the above-described triglycerides and optionally other components such as a partial glyceride, an antioxidant, an emulsifier, and a solvent such as water. The amount of these other components can be an optional amount unless the effects of the present invention are impaired. However, for example, in a case in which the mass of the obtained reactive substrate is 100% by mass, the amount is from 0% to 5% by mass, preferably from 0% to 2% by mass, and more preferably from 0% to 1% by mass.

The mixing may be carried out by any known mixing method as long as a homogeneous reactive substrate can be obtained. For example, the mixing may be carried out by using a paddle mixer, an AGI HOMO MIXER, a Disper mixer, or the like.

The mixing may be carried out under heating, as necessary. The heating temperature is preferably about the same as that in step (b) described below. For example, heating is carried out from 50° C. to 120° C., preferably from 60° C. to 100° C., more preferably from 70° C. to 90° C., and still more preferably 80° C. In the case of adding an enzyme as a catalyst, it is preferable that as little water as possible is present before the enzyme is added. The amount of water before the addition of the enzyme with respect to the total mass of raw materials is, for example, 10% by mass or less, preferably from 0.001% to 5% by mass, more preferably from 0.01% to 3% by mass, and still more preferably from 0.01% to 2% by mass, if appropriate. The mixing may last, for example, from 5 to 60 minutes, preferably from 10 to 50 minutes, and more preferably from 20 to 40 minutes.

### <Transesterification Reaction>

The transesterification reaction is caused in the presence of a catalyst by heating the above-described reactive substrate (mixture containing an XXX-type triglyceride and a YYY-type triglyceride), thereby obtaining a transesterification reaction product (oil or fat composition containing an XXX-type triglyceride and an X2Y-type triglyceride).

The transesterification reaction is not particularly limited, and any commonly used transesterification reaction can be used.

Here, the heating is carried out at, for example, from 50° C. to 120° C., preferably from 60° C. to 100° C., more preferably from 70° C. to 90° C., and still more preferably 80° C.

As the catalyst, an enzyme, an alkali metal alkoxide, an alkaline earth metal alkoxide, or the like can be used. As the enzyme, an immobilized enzyme or a powdered enzyme can be used, but from the viewpoints of enzyme activity and ease of handling, a powdered enzyme is preferred.

The powdered enzyme is prepared by drying and powdering an enzyme-containing aqueous liquid by methods such as spray drying, freeze drying, and drying after solvent precipitation. Examples thereof include, but are not particularly limited to, Alcaligenes sp.-derived lipase (trade name: Lipase QLM, Meito Sangyo Co., Ltd.).

The immobilized enzyme that can be used is an enzyme immobilized on a carrier such as silica, celite, diatomaceous earth, perlite, polyvinyl alcohol, anion exchange resin, phenol adsorption resin, hydrophobic carrier, cation exchange resin, or chelating resin.

In the alkali metal alkoxide and alkaline earth metal alkoxide that can be used as catalysts, lithium, sodium, potassium, or the like can be preferably used as the alkali metal. As the alkaline earth metal, magnesium and calcium can be preferably used. Examples of the alkoxide can include methoxide, ethoxide, propoxide, n-butoxide, and t-butoxide, with methoxide and ethoxide being preferred. Preferred examples of alkali metal alkoxides and alkaline earth metal alkoxides can include sodium methoxide, sodium ethoxide, magnesium methoxide, and magnesium ethoxide, with sodium methoxide being more preferred.

These catalysts may be used singly or in combination of two or more types, but it is preferable not to use an enzyme catalyst and an alkoxide catalyst at the same time.

The amount of the catalyst may be any amount that allows the transesterification reaction to proceed sufficiently. However, the amount added with respect to the total mass of triglycerides as raw materials is, for example, from 0.01% to 20% by mass, preferably from 0.05% to 10% by mass, more preferably from 0.1% to 5% by mass, and still more preferably from 0.2% to 1% by mass. In addition to the above catalysts, any promoter may be used.

The transesterification reaction is carried out, for example, under normal pressure or reduced pressure at the heating temperature described above, for example, for from 0.5 to 50 hours, preferably from 1 to 40 hours, more preferably from 5 to 30 hours, and still more preferably from 10 to 20 hours, optionally with stirring. In this reaction step, for example, the above-described predetermined amount of catalyst may be added all at once, or the predetermined amount of catalyst may be added in from 2 to 30 portions, preferably from 3 to 20 portions, and more preferably from 5 to 15 portions. The catalyst may be added immediately after the above-described step (a) or at intervals of from 1 to 2 hours after the first catalyst addition.

### (a) Step of Preparing Oil or Fat Composition II

Examples of the method of producing an oil or fat composition prepared in step (a) in the present invention can further include a method of simultaneously and directly synthesizing an XXX-type triglyceride and an X2Y-type triglyceride as shown below. In other words, in the preparation step II, to obtain an XXX-type triglyceride and an X2Y-type triglyceride, without synthesizing an XXX-type triglyceride and an YYY-type triglyceride separately for transesterification, raw materials for producing both triglycerides (fatty acid or fatty acid derivative and glycerin) are charged, for example, into a single reaction vessel and are synthesized simultaneously and directly. The production method thereof may be any of the following methods:
(iv) a method of directly esterifying glycerin with a fatty acid X having x carbon atoms and a fatty acid Y having y carbon atoms (direct ester synthesis); (v) a method of reacting a fatty acid alkyl (e.g., fatty acid methyl and fatty acid ethyl) in which a carboxyl group of a fatty acid X having x carbon atoms and a carboxyl group of a fatty acid Y having y carbon atoms are each bonded to an alkoxyl group with glycerin under basic or acidic catalytic conditions (ester exchange synthesis using a fatty acid alkyl group); and (vi) a method of reacting a fatty acid halide (e.g., fatty acid chloride and fatty acid bromide) in which the hydroxyl group of a carboxyl group of a fatty acid X having x carbon atoms and the hydroxyl group of a carboxyl group of a fatty acid Y having y carbon atoms are each substituted with a halogen with glycerin in the presence of a basic catalyst (acid halide synthesis).

The oil or fat composition of the present invention can be produced by any of the above-mentioned methods. However, from the viewpoint of ease of production, (iv) direct ester synthesis or (v) transesterification synthesis using a fatty acid alkyl is preferred, and (iv) direct ester synthesis is more preferred.

In the direct ester synthesis (iv) of the oil or fat composition of the present invention,

the production method is not limited within the range in which the XXX-type triglyceride and the X2Y-type triglyceride are present at a desired value in % by mass in the total triglyceride. However, to ensure that the desired triglyceride is generated in situ, it is preferable to carry out the reaction in two stages. In other words, a method in which in the first stage, glycerin is reacted with a fatty acid X having x carbon atoms and a fatty acid Y having y carbon atoms, and then in the second step, a fatty acid X having x carbon atoms is added and reacted, thereby yielding an oil or fat composition containing certain amounts of an XXX-type triglyceride and an X2Y-type triglyceride is preferred.

In the first stage in a two-stage reaction, the total molar amount of fatty acid Y and fatty acid X adjusted to a range in which the percent of the X2Y-type triglyceride in the total glyceride satisfies a desired value by mass is in a molar amount of preferably from 0.5 to 2.8, more preferably from 0.8 to 2.57, and most preferably from 1.1 to 2.2 per 1 mole of glycerin. This allows a fatty acid Y as a whole to be reliably esterified with glycerin and ultimately allows an X2Y-type glyceride to be generated more reliably in situ.

The reaction temperature in the direct ester synthesis of the oil or fat composition of the present invention may be any temperature at which the water produced by the esterification reaction can be removed ex situ, and it is preferably from 120° C. to 300° C., more preferably from 150° C. to 270° C., and still more preferably from 180° C. to 250° C. In particular, by carrying out the reaction at from 180° C. to 250° C., the X2Y-type triglyceride can be produced efficiently.

In the direct ester synthesis (iv) of the oil or fat composition of the present invention, a catalyst for promoting the esterification reaction may be used. Examples of the catalyst include an acid catalyst and an alkoxide of an alkaline earth metal. The amount of the catalyst used is preferably about from 0.001% to 1% by mass based on the total mass of the reaction raw materials.

In the direct ester synthesis (iv) of the oil or fat composition of the present invention, the catalyst and unreacted raw materials can be removed after the reaction by carrying out known purification treatments such as washing with water, alkali deoxidation and/or deoxidation under reduced pressure, and adsorption treatment. Furthermore, the reaction product obtained can be further purified via decolorization and deodorization treatment.

### (a) Step of Preparing Oil or Fat Composition III

Furthermore, the oil or fat composition may be obtained as an oil or fat composition containing from 80% to 99% by mass of an XXX-type triglyceride and from 20% to 1% by mass of an X2Y-type triglyceride by preparing an oil or fat composition containing an XXX-type triglyceride outside a range of from 80% to 99% by mass and/or an X2Y-type triglyceride outside a range of from 20% to 1% by mass and then further adding an XXX-type triglyceride or an X2Y-type triglyceride (preparation of an oil or fat composition by dilution). For example, an oil or fat composition containing from 80% to 99% by mass of an XXX-type triglyceride and from 20% to 1% by mass of an X2Y-type triglyceride may be obtained by obtaining an oil or fat composition containing from 50% to 70% by mass of an XXX-type triglyceride and from 50% to 30% by mass of an X2Y-type triglyceride and then adding a desired amount of an XXX-type triglyceride.

Furthermore, the above-described preparation step III also includes preparing an oil or fat composition containing an XXX-type triglyceride in a range of from 80% to 99% by mass and/or an X2Y-type triglyceride in a range of from 20% to 1% by mass in the above-described preparation step I or II, and then further adding an XXX-type triglyceride or an X2Y-type triglyceride, thereby adjusting the percent by mass of XXX-type triglyceride and the percent by mass of X2Y-type triglyceride within more preferred ranges (preparation of more suitable oil or fat composition by dilution).

### (b) Step of Obtaining the Oil or Fat Composition in Molten State

Prior to the above-described step (d), the oil or fat composition obtained in the above-described step (a) is cooled without being heated in a case in which it is in a molten state when being prepared. In a case in which it is not in a molten state when being obtained, the oil or fat composition is optionally heated for melting the triglycerides contained therein, thereby obtaining a molten oil or fat composition.

Here, the oil or fat composition is suitably heated to a temperature equal to or higher than the melting point of the triglycerides contained in the above-described oil or fat composition, in particular to a temperature capable of melting XXX-type and X2Y-type triglycerides, for example, from 70° C. to 200° C., preferably from 75° C. to 150° C., more preferably from 80° C. to 100° C. The heating is suitably continued for, for example, from 0.5 to 3 hours, preferably from 0.5 to 2 hours, and more preferably from 0.5 to 1 hour.

### (d) Step of Cooling Molten Oil or Fat Composition to Obtain Powdered Oil or Fat Composition

The molten oil or fat composition obtained in step (a) or (b) above is further cooled to form a powdered oil.

Here, “cooling the molten oil or fat composition” means keeping the molten oil or fat composition at a temperature lower than the melting point of the oil or fat composition. The “temperature lower than the melting point of the oil or fat composition” refers to, for example, a temperature 1° C. to 30° C. lower than the melting point, preferably a temperature 1° C. to 20° C. lower than the melting point, and more preferably a temperature 1° C. to 15° C. lower than the melting point. The molten oil or fat composition is cooled by performing cooling such that when x is, for example, from 8 to 10, the final temperature is preferably from 10° C. to 30° C., more preferably from 15° C. to 25° C., and still more preferably from 18° C. to 22° C. When x is, for example, 11 or 12, the final temperature in cooling is preferably from 30° C. to 40° C., more preferably from 32° C. to 38° C., and still more preferably from 33° C. to 37° C. At the above-described final temperature, it is appropriate to leave the molten oil or fat composition for, for example, preferably 2 hours or more, more preferably 4 hours or more, and further preferably 6 hours to 2 days. In particular, in a case in which the following step (c) is not used, it may be necessary to leave the molten oil or fat composition to stand for, for example, from 2 to 8 days, specifically from 3 to 7 days, and more specifically about 6 days.

### (c) Step of Promoting Powder Generation

Furthermore, between steps (a) or (b) and (d) above, as an optional step (c) of promoting powder generation, the molten oil or fat composition used in step (d) may be subjected to a seeding method (c1), a tempering method (c2), and/or a pre-cooling method (c3). These optional steps (c1) to (c3) may be carried out either singly or in combination of two or more steps. Here, between step (a) or (b) and step (d) means during step (a) or (b), after step (a) or (b) and before step (d), and during step (d).

The seeding method (c1) and the tempering method (c2) are methods of promoting powder generation in which a molten oil or fat composition is treated before being cooled to the final temperature so as to convert the molten oil or fat composition more reliably into a powder in the production of powdered oil or fat.

The seeding method is a method in which a small amount of a component that will become the core (seed) of powder is added to a molten oil or fat composition when the composition is cooled, thereby promoting powderization. Specifically, for example, an oil or fat powder containing preferably 80% by mass or more, and more preferably 90% by mass or more of an XXX-type triglyceride having the number of carbon atoms the same as in the XXX-type triglyceride in the oil or fat composition obtained in the molten state in step (b) is prepared as a core (seed) component. The method is a method of promoting the powderization of an oil or fat composition by adding the oil or fat powder serving as the core in an amount of from 0.1 to 1 part by mass and preferably from 0.2 to 0.8 parts by mass with respect to 100 parts by mass of the oil or fat composition in a molten state when cooling the molten oil or fat composition at a time when the temperature of the oil or fat composition reaches, for example, a final cooling temperature of from #0° C. to +10° C. and preferably from +5° C. to +10° C.

The tempering method is a method of promoting the powderization of an oil or fat composition by cooling an oil or fat composition in a molten state once to a temperature lower than the cooling temperature in step (d), for example, a temperature 5° C. to 20° C. lower, preferably a temperature 7° C. to 15° C. lower, more preferably about 10° C. lower, for preferably from 10 to 120 minutes, more preferably about from 30 to 90 minutes before allowing the oil or fat composition to stand at the final cooling temperature.
(c3) The pre-cooling method is a method in which the molten oil or fat composition obtained in step (a) or (b) is pre-cooled once at a temperature lower than the temperature of the molten state in step (a) or (b) and higher than the cooling temperature in step (d) before cooling in step (d). The temperature higher than the cooling temperature in step (d) may be, for example, a temperature that is 2° C. to 40° C. higher, preferably 3° C. to 30° C. higher, more preferably 4° C. to 30° C. higher, and still more preferably about 5° C. to 10° C. higher than the cooling temperature in step (d). The lower the pre-cooling temperature is set, the shorter the main cooling time at the cooling temperature in step (d) can be. In other words, unlike the seeding method and tempering method, the pre-cooling method is a method that can promote the powderization of an oil or fat composition simply by lowering the cooling temperature in stages and is, therefore, highly advantageous for industrial production.

### (e) Step of Pulverizing the Solid to Obtain Powdered Oil or Fat

More specifically, the step of obtaining a powdered oil or fat by cooling in step (d) above may be carried out by a step (e) of pulverizing the solid obtained by the cooling in step (d) to obtain a powdered oil or fat.

To explain in detail, first, the oil or fat composition containing the XXX-type triglyceride and the X2Y-type triglyceride described above is melted so as to obtain a molten oil or fat composition, which is then cooled so as to form a solid having voids and a volume increased compared to that of the molten oil or fat composition. The oil or fat composition which has become a solid having voids can be pulverized by applying a light impact, and the solid easily disintegrates into a powder form.

Here, the means for applying the light impact is not particularly specified, but a method of crushing (loosening) the composition by applying light vibration (impact) such as shaking or sieving is simple and preferable.

The content of the powdered oil or fat (A) is preferably from 0.5% by mass to 20% by mass, more preferably from 0.7% by mass to 15% by mass, still more preferably from 0.8% by mass to 12% by mass, and even more preferably from 1.0% by mass to 10% by mass with respect to the total mass of a powder cosmetic. As long as the content of the powdered oil or fat is within the above-described numerical range, many of the properties of the powder cosmetic when in use (particularly smoothness upon application, suppression of powdery feel upon application, and skin glow after application) become excellent.

### (B) Powder Component (Excluding the Powdered Oil or Fat (A))

The powder component (B) (excluding the powdered oil or fat (A)) is not particularly limited, and conventionally known powder cosmetic components can be used. Examples of the powder component include mica, synthetic phlogopite, silica, glass (borosilicate glass, soda lime glass, or the like), magnesium myristate, lauroyl lysine, talc, synthetic phlogopite iron, a cellulose powder, a urethane powder, a silicone resin powder, a polymethacrylic acid powder, titanium oxide, iron oxide, and a tar dye. These powder components may be used singly or in combination of two or more kinds. The shape of the powder component (B) is not particularly limited, but examples include spherical, cubic, flake, plate, rod, and needle shapes. Among these, a spherical shape is preferred.

The total content of the powdered oil or fat (A) and the powder component (B) is preferably from 90% by mass to 99.9% by mass, more preferably from 93% by mass to less than 99.5% by mass, still more preferably from 95% by mass to less than 99% by mass with respect to the total mass of the powder cosmetic. As long as the total content of the powdered oil or fat (A) and the powder component (B) is within the above-described numerical range, many of the properties of the powder cosmetic when in use (particularly smoothness upon application, suppression of powdery feel upon application, and skin glow after application) become excellent.

(C) Liquid Oil or Fat Incompatible with Powdered Oil or Fat (A)

As the liquid oil or fat that is incompatible with the above-described powdered oil or fat, a silicone oil can be suitably used.

Examples of a silicone oil include linear polysiloxanes (such as dimethylpolysiloxane, methylphenylpolysiloxane, and diphenylpolysiloxane), cyclic polysiloxanes (such as octamethylcyclotetrasiloxane, decamethylcyclopentasiloxane, and dodecamethylcyclohexasiloxane), silicone resin forming a three-dimensional network structure, silicone rubber, various modified polysiloxanes (such as amino-modified polysiloxanes, polyether-modified polysiloxanes, alkyl-modified polysiloxanes, and fluorine-modified polysiloxanes), and acrylic silicones. These silicone oils may be used singly or in combination of two or more kinds.

The content of the liquid oil or fat (C) that is incompatible with the powdered oil or fat (A) is not particularly limited. However, it is preferably from 0.1% by mass to 10% by mass, more preferably from 0.5% by mass to 7% by mass, and still more preferably from 1% by mass to 5% by mass with respect to the total mass of the powder cosmetic. As long as the content of the liquid oil or fat that is incompatible with the powdered oil or fat (A) is within the above-described numerical range, many of the properties of the powder cosmetic when in use (particularly smoothness upon application, suppression of powdery feel upon application, and skin glow after application) become excellent.

The blending ratio (A/C) of the component (A) (powdered oil or fat) to the component (C) (liquid oil or fat that is incompatible with the powdered oil or fat) in the powder cosmetic is preferably from 0.1 to 10, more preferably from 0.2 to 7, and still more preferably from 0.5 to 5 on a mass basis. As long as the blending ratio (A/C) is within the above-described numerical range, many of the properties of the powder cosmetic when in use (particularly smoothness upon application, suppression of powdery feel upon application, and skin glow after application) become excellent.

### (Other Components)

A cosmetic according to the present invention may contain, in addition to the above-described components, other components that can be blended into cosmetics, as long as the effects of the present invention are not impaired. Examples of such other components include an ultraviolet absorbing agent, a moisturizing agent, a dispersing agent, a neutralizing agent, a chelating agent, a preservative, a surface treatment agent, an antioxidant, a stabilizer, a coloring agent, and a fragrance. These components can be blended appropriately depending on the dosage form of the cosmetic.

Examples of the ultraviolet absorbing agent include: benzoic acid-based ultraviolet absorbing agents (such as para-aminobenzoic acid (hereinafter abbreviated as PABA), PABA monoglycerin ester, N,N-dipropoxy PABA ethyl ester, N, N-diethoxy PABA ethyl ester, N,N-dimethyl PABA ethyl ester, N, N-dimethyl PABA butyl ester, and N, N-dimethyl PABA ethyl ester); anthranilic acid-based ultraviolet absorbing agents (such as homomenthyl-N-acetylanthranilate); salicylic acid-based ultraviolet absorbing agents (such as amyl salicylate, menthyl salicylate, homomenthyl salicylate, octyl salicylate, phenyl salicylate, benzyl salicylate, and p-isopropanol phenyl salicylate); cinnamic acid-based UV absorbing agents (such as octyl cinnamate, ethyl-4-isopropyl cinnamate, methyl-2,5-diisopropyl cinnamate, ethyl-2,4-diisopropyl cinnamate, methyl-2,4-diisopropylcinnamate, propyl-p-methoxycinnamate, isopropyl-p-methoxycinnamate, isoamyl-p-methoxycinnamate, Octyl-p-methoxycinnamate (2-ethylhexyl-p-methoxycinnamate), 2-ethoxyethyl-p-methoxycinnamate, cyclohexyl-p-methoxycinnamate, ethyl-α-cyano-β-phenylcinnamate, 2-Ethylhexyl-α-cyano-β-phenylcinnamate, and glyceryl mono-2-ethylhexanoyl-di-para-methoxycinnamate); benzophenone-based ultraviolet absorbing agents (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone); 3-(4′-methylbenzylidene)-d,l-camphor, 3-benzylidene-d,l-camphor; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzalazine; dianisoylmethane; 4-methoxy-4′-t-butyldibenzoylmethane; and 5-(3,3-dimethyl-2-norbornylidene)-3-pentan-2-one.

The moisturizing agent is not particularly limited, and any moisturizing agent conventionally known for use in cosmetics can be used. Examples of such moisturizing agents include polyhydric alcohols and glycol ethers, more specifically, glycerin, propanediol, ethylene glycol, diethylene glycol, dipropylene glycol, 1,3-butylene glycol, hexamethylene glycol, isoprene glycol, polyethylene glycol, hyaluronic acid, xylitol, sorbitol, maltitol, and diglycerin (EO)PO adducts.

The powder cosmetic according to the present invention can be produced in accordance with an ordinary method, and the production method thereof is not particularly limited.

### (Intended Use)

The powder cosmetic according to the present invention can be suitably used as a general powder cosmetic such as loose powder, pressed powder, solid powder foundation, eye shadow, and the like. The powder cosmetic according to the present invention is particularly preferably used as a loose powder.

### EXAMPLES

The present invention will be specifically described based on the following examples, but the present invention is not limited to these examples. Unless otherwise specified, the contents are expressed in % by mass.

### Examples 1 to 4, Comparative Examples 1 to 5

### <Preparation of Powder Cosmetics>

Loose powders of the Examples and Comparative Examples were prepared according to the formulations listed in Tables 1 and 2. In Tables 1 and 2, the blending ratio of each component is expressed as % by mass.

The triglyceride composition of the powdered oil or fat used in each powder cosmetic was analyzed by the following method.

### [Analysis Method]

### Triglyceride Composition

### Gas Chromatographic Analysis Conditions

DB1-ht (0.32 mm×0.1 μm×5 m) from Agilent Technologies (123-1131)
Injection volume: 1.0 μL
Inlet: 370° C.
Detector: 370° C.
Split ratio: 50/1 35.1 kPa constant pressure
Column CT: 200° C. (0 min hold)—(15° C./min)—370° C. (4 min hold)

### <Performance Evaluation of Powder Cosmetics>

The usability of each of the powder cosmetics prepared above was evaluated. Specifically, a panel of 10 experts who were trained in sensory evaluation and capable of evaluating according to certain standards applied the samples to their faces and evaluated the following properties using the results of Comparative Example 1 as the standards. The evaluation results are shown in Tables 1 and 2.

### [Smoothness Upon Application]

A: Compared to the standard, the smoothness upon application was extremely superior.
B: Compared to the standard, the smoothness upon application was superior.
C: Compared to the standard, the smoothness upon application was slightly superior.
D: The smoothness upon application was equivalent to the standard.
E: Compared to the standard, the smoothness upon application was inferior.

### [Suppression of Powdery Feel Upon Application]

A: Compared to the standard, the suppression of powdery feel upon application was extremely superior.
B: Compared to the standard, the suppression of powdery feel upon application was superior.
C: Compared to the standard, the suppression of powdery feel upon application was slightly superior.
D: The suppression of powdery feel upon application was equivalent to the standard.
E: Compared to the standard, the suppression of powdery feel upon application was inferior.
   [Skin Glow after Application]
A: Compared to the standard, the skin glow after application was extremely superior.
B: Compared to the standard, the skin glow after application was superior.
C: Compared to the standard, the skin glow after application was slightly superior.
D: The skin glow after application was equivalent to the standard.
E: Compared to the standard, the skin glow after application was inferior.

### [Resistance to Granulation]

A: Compared to the standard, the resistance to granulation was extremely superior.
B: Compared to the standard, the resistance to granulation was superior.
C: Compared to the standard, the resistance to granulation was slightly superior.
D: The resistance to granulation was equivalent to the standard.
E: Compared to the standard, the resistance to granulation was inferior.

TABLE 1

| Composition of Powder cosmetic | | Description | Example 1 | Example 2 | Example 3 | Example 4 |
|---|---|---|---|---|---|---|
| A | Powdered oil or fat *1 | Powder | 5 | 5 | 10 | 1 |
| B | Mica | Powder | 35 | 35 | 35 | 35 |
| B | Synthetic phlogopite | Powder | 31.43 | 32.43 | 26.43 | 35.43 |
| B | Borosilicate glass (Ca/Al) | Powder | 10 | 10 | 10 | 10 |
| B | Magnesium myristate | Powder | 5 | 5 | 5 | 5 |
| B | Lauroyl lysine | Powder | 2 | 2 | 2 | 2 |
| B | Silicone resin powder | Powder | 3 | 3 | 3 | 3 |
| B | Color material | Powder | 0.37 | 0.37 | 0.37 | 0.37 |
| B | Shiny powder (pearl agent) | Powder | 5 | 5 | 5 | 5 |
| C | Dimethicone (6cs) | Liquid | 2 | 2 | 2 | 2 |
| | Tri(caprylic acid/capric acid) glyceryl | Liquid | 0 | 0 | 0 | 0 |
| | Ethylhexyl methoxycinnamate | Liquid | 0 | 0 | 0 | 0 |
| | Distearyldimonium chloride | — | 1 | 0 | 1 | 1 |
| | Preservative | — | 0.2 | 0.2 | 0.2 | 0.2 |
| | | Total | 100 | 100 | 100 | 100 |
| | Component A | Subtotal of | 5 | 5 | 10 | 1 |
| | Component B | each | 91.8 | 92.8 | 86.8 | 95.8 |
| | Component C | component | 2 | 2 | 2 | 2 |
| | Component A/Component C | Mass basis | 2.5 | 2.5 | 5.0 | 0.5 |
| | Smoothness upon application | Evaluation | A | A | A | B |
| | Suppression of powdery feel upon | items | B | B | A | D |
| | application | | | | | |
| | Skin glow after application | | B | B | A | C |
| | Resistance to granulation | | D | D | D | D |

TABLE 2

| | | | Comparative | Comparative | Comparative | Comparative | Comparative |
|---|---|---|---|---|---|---|---|
| Composition of Powder cosmetic | | Description | Example 1 | Example 2 | Example 3 | Example 4 | Example 5 |
| A | Powdered oil or fat *1 | Powder | 0 | 5 | 0 | 1 | 1 |
| B | Mica | Powder | 35 | 35 | 35 | 35 | 35 |
| B | Synthetic phlogopite | Powder | 36.43 | 33.43 | 38.43 | 35.43 | 35.43 |
| B | Borosilicate glass (Ca/Al) | Powder | 10 | 10 | 10 | 10 | 10 |
| B | Magnesium myristate | Powder | 5 | 5 | 5 | 5 | 5 |
| B | Lauroyl lysine | Powder | 2 | 2 | 2 | 2 | 2 |
| B | Silicone resin powder | Powder | 3 | 3 | 3 | 3 | 3 |
| B | Color material | Powder | 0.37 | 0.37 | 0.37 | 0.37 | 0.37 |
| B | Shiny powder (pearl agent) | Powder | 5 | 5 | 5 | 5 | 5 |
| C | Dimethicone (6cs) | Liquid | 2 | 0 | 0 | 0 | 0 |
| | Tri(caprylic acid/capric acid) | Liquid | 0 | 0 | 0 | 2 | 0 |
| | glyceryl | | | | | | |
| | Ethylhexyl methoxycinnamate | Liquid | 0 | 0 | 0 | 0 | 2 |
| | Distearyldimonium chloride | — | 1 | 1 | 1 | 1 | 1 |
| | Preservative | — | 0.2 | 0.2 | 0.2 | 0.2 | 0.2 |
| | | Total | 100 | 100 | 100 | 100 | 100 |
| | Component A | Subtotal of | 0 | 5 | 0 | 1 | 1 |
| | Component B | each | 96.8 | 93.8 | 98.8 | 95.8 | 95.8 |
| | Component C | component | 2 | 0 | 0 | 0 | 0 |
| | Component A/Component C | Mass basis | 0 | — | — | — | — |
| | Smoothness upon application | Evaluation | D (Std) | E | E | E | E |
| | Suppression of powdery feel | items | D (Std) | B | E | D | D |
| | upon application | | | | | | |
| | Skin glow after application | | D (Std) | B | D | D | D |
| | Resistance to granulation | | D (Std) | D | D | E | E |

*Std: The standard for each evaluation.

*1: Trade name: Enequick manufactured by The Nisshin OilliO Group, Ltd. (the content of one or more XXX-type triglycerides having a fatty acid residue X having 10 carbon atoms at positions 1 to 3 is 93.9% by mass, and the content of X2Y-type triglycerides in which one of the fatty acid residues X in the XXX-type triglycerides is substituted with a fatty acid residue Y having 14 carbon atoms is 5.4% by mass with respect to a total triglyceride content of 100% by mass; melting point (peak top value on DSC chart): 28.5° C.)

From the above results, it can be seen that all of the powder cosmetics according to the present invention are excellent in terms of the smoothness upon application, the suppression of powdery feel upon application, and the skin glow after application. In addition, in consideration of these results with the results of the Comparative Examples, it can be seen that the powder cosmetic according to the present invention achieves excellent effects by blending a specific powdered oil or fat, powder components other than the powdered oil or fat, and a liquid oil or fat that is incompatible with the powdered oil or fat.

### <Formulation Example of Powder Cosmetic>

The ingredients listed below were mixed, thereby preparing a powder cosmetic. The following values are given in % by mass.

### (Ingredients)

| | |
|---|---|
| (A) Powdered oil or fat *1 (powder) | 1 |
| (B) Mica (powder) | 35 |
| (B) Synthetic phlogopite (powder) | 35.43 |
| (B) Borosilicate glass (Ca/Al) (powder) | 10 |
| (B) Magnesium myristate (powder) | 5 |
| (B) Lauroyl lysine (powder) | 2 |
| (B) Silica (powder) | 3 |
| (B) Color material (powder) | 0.37 |
| (B) Shiny powder (pearl agent) (powder) | 5 |
| (C) Dimethicone (6cs) (liquid) | 2 |
| Distearyldimonium chloride (powder) | 1 |
| Preservative (powder) | 0.2 |
| Total | 100 |

According to the above formulation example, it is possible to produce a powder cosmetic that is excellent in the smoothness upon application, the suppression of powdery feel upon application, and the skin glow after application. As a result, the range of cosmetics can be expanded.

1. A powder cosmetic comprising:
(A) a powdered oil or fat containing from 80% to 99% by mass of one or more XXX-type triglycerides having a fatty acid residue X having x carbon atoms at positions 1 to 3 and from 20% to 1% by mass of one or more X2Y-type triglycerides in which one of the fatty acid residues X in the XXX-type triglycerides is substituted with a fatty acid residue Y having y carbon atoms with respect to the total triglyceride content of 100% by mass, wherein the number of carbon atoms x is an integer selected from 8 to 12, and the number of carbon atoms y is each independently an integer selected from x+2 to x+8;
(B) a powder component (excluding the powdered oil or fat (A)); and
(C) a liquid oil or fat that is incompatible with the powdered oil or fat (A).

2. The powder cosmetic according to claim 1, wherein the powdered oil or fat (A) contains from 90% to 99% by mass of an XXX-type triglyceride having a fatty acid residue X having x carbon atoms at positions 1 to 3 and from 10% to 1% by mass of one or more X2Y-type triglycerides in which one of the fatty acid residues X in the XXX-type triglyceride is substituted with a fatty acid residue Y having y carbon atoms with respect to the total triglyceride content of 100% by mass, the number of carbon atoms x is 10, and the number of carbon atoms y is each independently an integer selected from x+4 to x+8.

3. The powder cosmetic according to claim 1, wherein the powdered oil or fat (A) has a melting point of from 25° C. to 50° C.

4. The powder cosmetic according to claim 1, wherein the content of the powdered oil or fat (A) is from 0.5% by mass to 20% by mass with respect to a total mass of the powder cosmetic.

5. The powder cosmetic according to claim 1, wherein the content of the liquid oil or fat (C) is from 0.1% by mass to 10% by mass with respect to a total mass of the powder cosmetic.

6. The powder cosmetic according to claim 1, wherein a blending ratio (A/C) of the powdered oil or fat (A) to the liquid oil or fat (C) is from 0.1 to 10 on a mass basis.

7. The powder cosmetic according to claim 1, wherein the total content of the powdered oil or fat (A) and the powder component (B) is from 90% by mass to 99.9% by mass with respect to a total mass of the powder cosmetic.

8. The powder cosmetic according to claim 1, wherein the liquid oil or fat (C) is a silicone oil.

9. The powder cosmetic according to claim 1, wherein the powder component (B) contains at least one selected from the group consisting of mica, synthetic phlogopite, silica, glass, magnesium myristate, lauroyl lysine, talc, synthetic phlogopite iron, a cellulose powder, a urethane powder, a silicone resin powder, a polymethacrylic acid powder, titanium oxide, iron oxide, and a tar dye.

10. The powder cosmetic according to claim 1, wherein the powder cosmetic is a loose powder.
